(19) United States
(12) Patent Application Publication
(10) Pub. No.: US 2026/0057655 A1
(43) Pub. Date: Feb. 26, 2026

(54) HUMAN FACTOR INTELLIGENCE-BASED VITAL SIGN SIGNAL MEASUREMENT METHOD AND APPARATUS, AND DEVICE

(71) Applicant: KINGFAR INTERNATIONAL INC., Beijing (CN)

(72) Inventors: Qichao ZHAO, Beijing (CN); Qingju WANG, Beijing (CN)

(21) Appl. No.: 19/373,783

(22) Filed: Oct. 30, 2025

Related U.S. Application Data
(63) Continuation of application No. PCT/CN2024/141062, filed on Dec. 20, 2024.

(30) Foreign Application Priority Data
Dec. 22, 2023 (CN) 202311792773.3
Dec. 25, 2023 (CN) 202311801230.3

(51) Int. Cl. G06V 10/80; A61B 5/0205; A61B 5/024; G06F 21/32; G06V 10/25; G06V 10/764; G06V 10/77; G06V 20/40; G06V 20/52

(52) U.S. Cl. CPC G06V 10/806; A61B 5/0205; A61B 5/02416; G06F 21/32; G06V 10/25; G06V 10/764; G06V 10/7715; G06V 20/46; G06V 20/52

(57) ABSTRACT

Embodiments of the present disclosure provide a human factor intelligence-based vital sign signal measurement method and apparatus, and a device. The method includes: obtaining individual feature representation data of a measured object, environmental feature representation data of an environment where the measured object is located, and vital sign spectrum data of the measured object, wherein the individual feature representation data and the environmental feature representation data have a differential impact on the vital sign spectrum data; and performing a signal value prediction based on the individual feature representation data, the environmental feature representation data, and the vital sign spectrum data to obtain a vital sign signal value with the differential impact removed.

### CROSS-REFERENCE TO RELATED APPLICATIONS

This application is a continuation of International Patent Application No. PCT/CN2024/141062 filed on Dec. 20, 2024, which claims priorities to Chinese Patent Application No. 202311792773.3, titled “HUMAN FACTOR INTELLIGENCE-BASED VITAL SIGN SIGNAL MEASUREMENT METHOD AND APPARATUS, AND DEVICE” and filed with China National Intellectual Property Administration on Dec. 22, 2023, and Chinese Patent Application No. 202311801230.3, titled “HUMAN FACTOR INTELLIGENCE-BASED VITAL SIGN SIGNAL DETECTION METHOD AND RELATED DEVICE” and filed with China National Intellectual Property Administration on Dec. 25, 2023, the entire disclosures of which are incorporated herein by reference.

### FIELD

Embodiments of the present disclosure relate to the field of computer technologies, and more particularly, to a human factor intelligence-based vital sign signal measurement method and apparatus, and a device.

### BACKGROUND

Vital sign signals are a set of medical parameters that can describe health states and bodily functions of individuals, which may include a heart rate, a respiratory rate, a body temperature, and a blood pressure, etc.

In the related art, when measuring the vital sign signals, measurements can typically be made using contact sensors such as electrocardiogram monitors, or based on millimeter-wave radar. However, using the contact sensors for the measurement may interfere with vital sign signals of a measured object and require regular calibration and maintenance, imposing certain limitations. While millimeter-wave radar-based vital sign signal measurement is susceptible to interference and its measurement accuracy needs to be improved.

### SUMMARY

The present disclosure provides a human factor intelligence-based vital sign signal measurement method and apparatus, and a device, to improve measurement accuracy of the vital sign signals.

Embodiments of the present disclosure provide a human factor intelligence-based vital sign signal measurement method. The method includes: obtaining individual feature representation data of a measured object, environmental feature representation data of an environment where the measured object is located, and vital sign spectrum data of the measured object, wherein the individual feature representation data and the environmental feature representation data have a differential impact on the vital sign spectrum data; and performing a signal value prediction based on the individual feature representation data, the environmental feature representation data, and the vital sign spectrum data to obtain a vital sign signal value with the differential impact removed.

Embodiments of the present disclosure provide a human factor intelligence-based vital sign signal measurement apparatus. The apparatus includes a feature data obtaining module and a vital sign determination module. The feature data obtaining module is configured to obtain individual feature representation data of a measured object, environmental feature representation data of an environment where the measured object is located, and vital sign spectrum data of the measured object, wherein the individual feature representation data and the environmental feature representation data have a differential impact on the vital sign spectrum data. The vital sign determination module is configured to perform a signal value prediction based on the individual feature representation data, the environmental feature representation data, and the vital sign spectrum data to obtain a vital sign signal value with the differential impact removed.

Embodiments of the present disclosure provide a computer device. The computer device includes a memory and a processor. The memory has a computer program stored thereon. The processor is configured to implement, when executing the computer program, the vital sign signal measurement method according to any one of the embodiments described above.

Embodiments of the present disclosure provide an edge computing device. The edge computing device includes a memory, a processor, and a communication interface. The memory has a computer program stored thereon. The processor is configured to implement, when executing the computer program, the vital sign signal measurement method described above.

Embodiments of the present disclosure provide a computer-readable storage medium. The computer-readable storage medium has a computer program stored thereon. The computer program is configured to implement, when executed by a processor, the vital sign signal measurement method according to any one of the embodiments described above.

In the embodiments of the present disclosure, the individual feature representation data of the measured object, the environmental feature representation data of the environment where the measured object is located, and the vital sign spectrum data of the measured object are obtained, and the signal value prediction is performed based on the individual feature representation data, the environmental feature representation data, and the vital sign spectrum data, to obtain the vital sign signal value with the differential impact of each of the individual feature representation data and the environmental feature representation data on the vital sign spectrum data removed. In this way, the measurement accuracy of the vital sign signals is improved.

### BRIEF DESCRIPTION OF THE DRAWINGS

FIG. 1a is a schematic diagram of a vital sign signal measurement system according to an embodiment of the present disclosure.

FIG. 1b is a schematic diagram of a vital sign signal measurement system according to an embodiment of the present disclosure.

FIG. 2 is a schematic flowchart of a vital sign signal measurement method according to an embodiment of the present disclosure.

FIG. 3 is a schematic flowchart of a method for obtaining individual feature representation data according to an embodiment of the present disclosure.

FIG. 4 is a schematic flowchart of a method for obtaining environmental feature representation data according to an embodiment of the present disclosure.

FIG. 5 is a schematic flowchart of a method for obtaining vital sign spectrum data according to an embodiment of the present disclosure.

FIG. 6 is a schematic flowchart of a method for determining a target range bin according to an embodiment of the present disclosure.

FIG. 7a is a schematic flowchart of a vital sign signal measurement method according to an embodiment of the present disclosure.

FIG. 7b is a schematic flowchart of a method for obtaining environmental feature representation data according to an embodiment of the present disclosure.

FIG. 8a is a schematic flowchart of a method for training a target classification model according to an embodiment of the present disclosure.

FIG. 8b is a schematic flowchart of a method for constructing a training sample according to an embodiment of the present disclosure.

FIG. 9 is a schematic diagram of a computer device according to an embodiment of the present disclosure.

### DETAILED DESCRIPTION OF THE EMBODIMENTS

In order for those skilled in the art to better understand solutions of the present disclosure, reference will be made clearly and completely to technical solutions in the embodiments of the present disclosure with accompanying drawings. Obviously, the embodiments described here are only part of the embodiments of the present disclosure and are not all embodiments of the present disclosure. Based on the embodiments of the present disclosure, other embodiments obtained by those skilled in the art without creative labor are within scope of the present disclosure.

In some related arts, when measuring the vital sign signals, measurements can typically be made using contact sensors such as electrocardiogram monitors, or based on millimeter-wave radar. However, using the contact sensors for the measurement may interfere with vital sign signals of a measured object and require regular calibration and maintenance, imposing certain limitations. While millimeter-wave radar-based vital sign signal measurement is susceptible to interference and its measurement accuracy needs to be improved.

Therefore, during the millimeter-wave radar-based vital sign signal measurement, it is necessary to provide a human factor intelligence-based vital sign signal measurement method, i.e., an individualized vital sign signal measurement method, to measure vital sign signals for different measured objects. Firstly, the individual feature representation data of the measured object, the environmental feature representation data of the environment where the measured object is located, and the vital sign spectrum data of the measured object are obtained. Next, signal value prediction is performed based on the individual feature representation data, the environmental feature representation data, and the vital sign spectrum data, to obtain the vital sign signal value with the differential impact of each of the individual feature representation data and the environmental feature representation data on the vital sign spectrum data removed. In this way, accuracy when measuring the vital sign signal of the measured object can be improved.

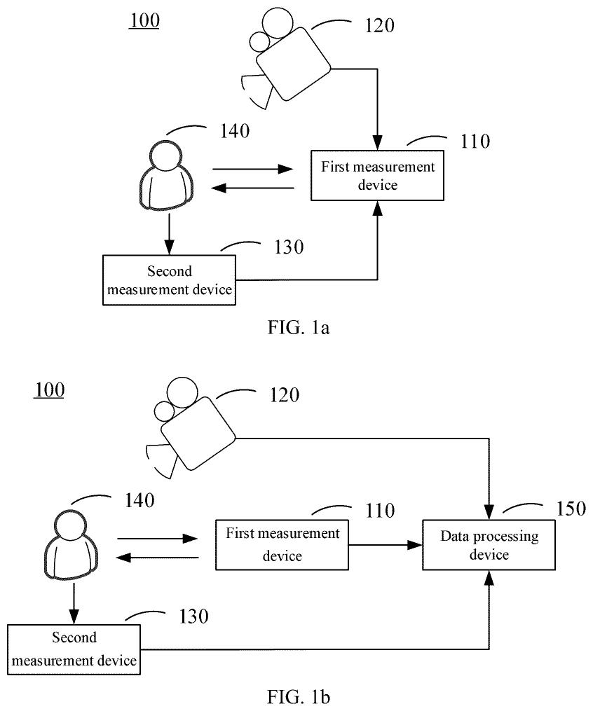

Referring to FIG. 1a, FIG. 1a is a schematic diagram of a scenario example of a human factor intelligence-based vital sign signal measurement system according to an embodiment of the present disclosure. A vital sign signal measurement system 100 may include a first measurement device 110, a video signal collection device 120, and a second measurement device 130.

In an embodiment, the first measurement device 110 is configured to transmit and receive a frequency-modulated continuous-wave radar signal. As a result, a digital mixing signal can be determined based on the transmission and the reception of the frequency-modulated continuous-wave radar signal. Thus, vital sign spectrum data of a measured object 140 can be obtained based on the digital mixing signal.

In an embodiment, the video signal collection device 120 may have a depth camera or a multi-view matching camera. For example, the multi-view matching camera may be a binocular matching camera. The video signal collection device 120 is configured to capture an environment where the measured object 140 is located through the depth camera or the multi-view matching camera to obtain a video signal. As a result, biometric feature data of the measured object and environmental data of the environment where the measured object can be obtained based on the video signal. Thus, individual feature representation data of the measured object can be obtained based on the biometric feature data, and environmental feature representation data of the environment where the measured object is located can be obtained based on the environmental data.

In an embodiment, the second measurement device 130 may be a measurement device that differs from the first measurement device 110. Exemplarily, the second measurement device 130 may be a contact measurement device or a contact sensor. As an example, the second measurement device 130 may be any one of a mechanical measurement device and a biological signal measurement device. The mechanical measurement device may be a respiratory belt sensor. For example, the respiratory belt sensor may be fixed to a chest using an elastic belt or a thoracic belt and configured to measure a respiratory rate by detecting expansion and contraction changes of each of the elastic belt and the thoracic belt. The mechanical measurement device may also be a respiratory mass sensor. For example, the respiratory mass sensor can be configured to measure a flow velocity and a flow volume of respiratory gas using a pressure sensor or a mass sensor and calculate the respiratory rate by analyzing a characteristic of the respiratory gas. The biological signal measurement device may be an electrocardiogram (ECG) monitoring device or a peripheral oxygen saturation (SpO2) monitoring device. The ECG monitoring device can be configured to collect an electrocardiogram signal through an electrode attached to a skin to calculate a heart rate. The SpO2 monitoring device can be configured to measure a peripheral oxygen saturation in blood through a photoelectric sensor, to calculate a heart rate and a respiratory rate. In this way, a truth value for training a target classification model can be obtained by the second measurement device.

As an example, the first measurement device 110 may be connected to the video signal collection device 120 and the second measurement device 130. The first measurement device 110 may be deployed with a target classification model to determine a vital sign signal value through the target classification model. The target classification model deployed at the first measurement device 110 is obtained through model training based on vital sign spectrum data, individual feature representation data, environmental feature representation data, and a truth value at the same historical moment.

As another example, referring to FIG. 1b, FIG. 1b is a schematic diagram of a scenario example of a vital sign signal measurement system according to an embodiment of the present disclosure. The vital sign signal measurement system 100 may further include a data processing device 150. The data processing device 150 may be connected to the first measurement device 110, the video signal collection device 120, and the second measurement device 130. The data processing device 150 may be deployed with a target classification model to determine a vital sign signal value through the target classification model. The target classification model deployed at the data processing device 150 is obtained through model training based on vital sign spectrum data, individual feature representation data, environmental feature representation data, and a truth value at the same historical moment. Exemplarily, the data processing device 150 may be a server or a terminal.

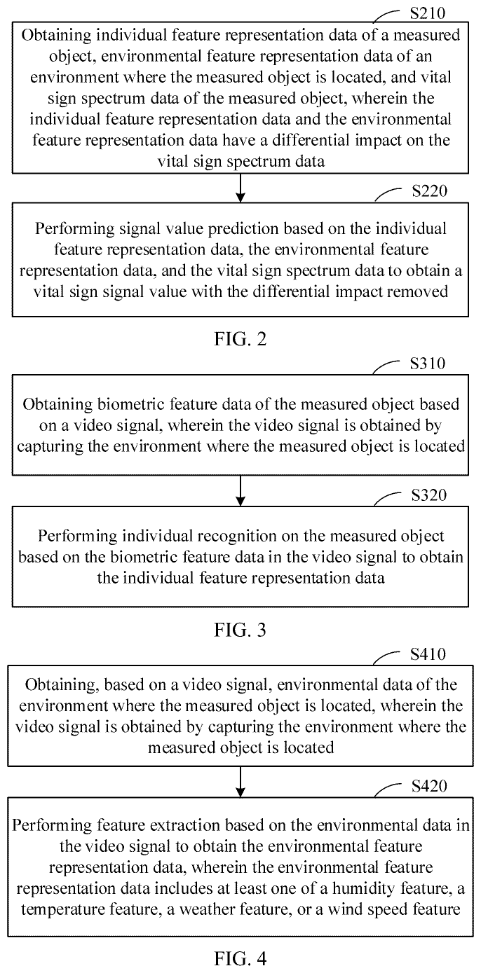

An embodiment of the present disclosure provides a human factor intelligence-based vital sign signal measurement method. Referring to FIG. 2, FIG. 2 is a schematic flowchart of a human factor intelligence-based vital sign signal measurement method according to this embodiment. This embodiment provides method operation steps as illustrated in the flowchart, and may include more or fewer operation steps based on conventional or non-creative efforts. A sequence of the steps listed in the embodiments is merely one of a plurality of step execution sequences, and does not represent a unique execution sequence. When executed by a system or a server product in practice, the steps may be executed sequentially or in parallel (e.g., a parallel processor or a multithread processing environment) based on the method sequence illustrated in the embodiments. The vital sign signal measurement method can be applied in the first measurement device or the data processing device in the vital sign signal measurement system. As shown in FIG. 2, the vital sign signal measurement method may include actions at steps 210 and 220.

At step S210, individual feature representation data of a measured object, environmental feature representation data of an environment where the measured object is located, and vital sign spectrum data of the measured object are obtained. The individual feature representation data and the environmental feature representation data have a differential impact on the vital sign spectrum data.

In some cases, the vital sign spectrum data corresponding to the measured object can be determined. In addition, the individual feature representation data of the measured object as well as the environmental feature representation data of the environment where the measured object is located can also be determined. As a result, the vital sign signal value of the measured object can be predicted based on the individual feature representation data, the environmental feature representation data, and the vital sign spectrum data.

The vital sign spectrum data may be determined based on millimeter-wave radar. For example, the first measurement device may be configured with a millimeter-wave radar, allowing the vital sign spectrum data to be determined based on the millimeter-wave radar of the first measurement device.

In an embodiment, frequency mixing can be performed based on a frequency-modulated continuous-wave radar signal transmitted and received by the first measurement device through the millimeter-wave radar to obtain a digital mixing signal, and the vital sign spectrum data of the measured object can be determined based on the digital mixing signal. The digital mixing signal is equivalent to a radio-frequency reflection signal in subsequent Embodiment 2, both of which are obtained by the millimeter-wave radar. The vital sign spectrum data of the measured object is equivalent to first feature data in Embodiment 2. Exemplarily, the first measurement device may generate the frequency-modulated continuous-wave radar signal through a millimeter-wave radar transmitter. A frequency of the frequency-modulated continuous-wave radar signal may vary over time, for example, increasing or decreasing over time within a specified time period. The transmitted frequency-modulated continuous-wave radar signal can be reflected back to the first measurement device by the measured object. The first measurement device may receive the reflected back frequency-modulated continuous-wave radar signal through the millimeter-wave radar receiver, and perform a mixing operation on the transmitted frequency-modulated continuous-wave radar signal and the received frequency-modulated continuous-wave radar signal to obtain a mixing signal. The mixing signal, i.e., an intermediate frequency signal or a beat signal, can facilitate subsequent determination of the vital sign spectrum data of the measured object.

Exemplarily, the frequency-modulated continuous-wave radar signal may be a chirp signal (Chirp).

Exemplarily, analog-to-digital conversion may also be performed on the mixing signal to obtain a digital mixing signal, and thus a subsequent spectral estimation-related operation can be facilitated based on the digital mixing signal.

Exemplarily, after determining the digital mixing signal, the spectrum estimation-related operation may be performed based on the digital mixing signal to determine the vital sign spectrum data. The spectral estimation-related operation may include at least one of the following: range determination, range bin determination, phase extraction, phase unwrapping, phase differencing, bandpass filtering, and spectral estimation.

Range determination (Range Fast Fourier Transform (FFT)) refers to performing the FFT on the digital mixing signal to determine range information, or performing the FFT on the digital mixing signal to determine a range curve, i.e., a spectrum of a range dimension determined by performing the FFT on the digital mixing signal. Thus, a plurality of range bins can be determined. Range bin determination (Range bin tracking) refers to determining a range bin corresponding to the measured object from the plurality of range bins. Phase extraction (extract phase) refers to extracting a phase of the range bin corresponding to the measured object. Phase unwrapping refers to perform phase unwrapping to obtain a phase signal. Phase differencing (Phase difference) refers to enhancing the unwrapped phase signal and reducing an existing phase drift. Bandpass filtering refers to filtering, based on different vital sign signals, corresponding phases in the phase signal using a bandpass filter for differentiation, or filtering frequencies formed by corresponding phase changes in the phase signal for differentiation, facilitating subsequent determination of different vital sign signals. Spectral estimation refers to performing the FFT on the obtained phase signal to obtain the vital sign spectrum data of the measured object. Thus, the vital sign signal of the measured object is determined based on the vital sign spectrum data of the measured object.

The individual feature representation data may be data that can describe the measured object or may be data used to recognize the measured object.

The environmental feature representation data may be data that can describe the environment where the measured object is located, or may be data used to recognize the environment where the measured object is located.

The differential impact of the individual feature representation data on the vital sign spectrum data may refer to an impact of an individual difference of the measured object on the vital sign spectrum data. For example, the individual difference of the measured object may refer to an identity, an age, a gender, a skin type, etc. of the measured object. For example, there may be a significant difference between a frequency-modulated continuous-wave radar signal reflected based on the skin type of oily skin and a frequency-modulated continuous-wave radar signal reflected based on the skin type of dry skin, resulting in a substantial difference in vital sign spectrum data obtained therefrom. Different skin types have different impacts on the difference in the vital sign spectrum data. That is, the individual feature representation data of the measured object has a differential impact on the vital sign spectrum data.

The differential impact of the environmental feature representation data on the vital sign spectrum data may refer to an impact of an environmental difference in the environment where the measured object is located on the vital sign spectrum data. For example, the environmental difference in the environment where the measured object is located may refer to a difference in light, brightness, temperature, and humidity in the environment where the measured object is located. For example, there is a significant difference between a frequency-modulated continuous-wave radar signal reflected when the environment where the measured object is located is a high-humidity environment and a frequency-modulated continuous-wave radar signal reflected when the environment of the measured object is located is a low-humidity environment. In the high-humidity environment, performance of the frequency-modulated continuous-wave radar signal may attenuate, which in turn may affect the determination of the vital sign signal of the measured object based on the vital sign spectrum data.

At step S220, signal value prediction is performed based on the individual feature representation data, the environmental feature representation data, and the vital sign spectrum data to obtain a vital sign signal value with the differential impact removed.

In the embodiments describe above, the individual feature representation data of the measured object, the environmental feature representation data of the environment where the measured object is located, and the vital sign spectrum data of the measured object are obtained, and the signal value prediction is performed based on the individual feature representation data, the environmental feature representation data, and the vital sign spectrum data, to remove the differential impact of each of the individual feature representation data and the environmental feature representation data on the vital sign spectrum data and obtain the corresponding vital sign signal value. In this way, the accuracy of performing the vital sign signal measurement on the measured object is improved.

In some embodiments, referring to FIG. 3, the obtaining the individual feature representation data of the measured object may include actions at steps S310 and S320.

At step S310, biometric feature data of the measured object is obtained based on a video signal. The video signal is obtained by capturing the environment where the measured object is located.

In some cases, the video signal collection device can be configured to capture the environment where the measured object is located and collect the video signal.

In an embodiment, the biometric feature data of the measured object can be obtained based on the video signal captured by the video signal collection device. Exemplarily, the biometric feature data of the measured object may be any one of facial data, iris data, retinal data, or eyeprint data of the measured object.

As an example, when obtaining the facial data based on the video signal captured by the video signal collection device, the video signal may be preprocessed. For example, preprocessing operations such as noise removal and contrast enhancement may be performed on the video signal to enhance quality of the video signal. After preprocessing the video signal, a facial region of the measured object may be recognized for the preprocessed video signal. When the facial region is detected, a facial feature can be further extracted. For example, facial features or facial data, such as a geometric feature and a texture feature, of a face, such as a facial contour, eyes, a mouth, and a nose, can be extracted, and thus subsequent individual recognition of the measured object can be facilitated based on the facial data.

As an example, the video signal collection device may be a designated device capable of performing any one of iris recognition, retinal recognition, or eyeprint recognition. As a result, the iris data, the retinal data, or the eyeprint data of the measured object can be obtained based on a video signal captured by the designated video signal collection device.

At step S320, individual recognition is performed on the measured object based on the biometric feature data in the video signal to obtain the individual feature representation data.

In an embodiment, biometric feature comparison data may be prestored; and an individual recognition result can be obtained by comparing biometric feature comparison data with the prestored biometric feature comparison data, and the individual feature representation data can be determined based on the individual recognition result.

The individual recognition result may include at least one of identity identification, a gender feature, an age feature, or a skin type feature of the measured object. Exemplarily, the identity identification may refer to identification information for uniquely identifying the measured object.

In some cases, the individual recognition result may be a high-dimensional sparse feature vector. In order to facilitate subsequent prediction of the vital sign signal value of the measured object, embedding processing can be performed on the individual recognition result to generate a low-dimensional dense feature vector of a predetermined dimension to obtain individual feature representation data. As a result, a computational load and storage space can be reduced during the prediction of the vital sign signal value based on the individual feature representation data, thereby improving prediction efficiency.

The high-dimensional sparse feature vectors can be used to process some unstructured data. For example, the individual recognition result of the measured object can be represented as a high-dimensional sparse feature vector. The low-dimensional dense feature vector, i.e., the individual feature representation data, may refer to the individual feature representation data obtained by the individual recognition result subjected to the embedding. It can retain an important feature and information of the individual recognition result as the high-dimensional sparse feature vector while reducing dimensionality and complexity of the individual recognition result.

In the embodiment described above, the biometric feature data of the measured object is obtained based on the video signal, and the individual recognition is performed on the measured object based on the biometric feature data to obtain individual feature representation data. In this way, the individual feature representation data for recognizing the measured object can be quickly obtained, which facilitates subsequent removal of the differential impact of the individual feature representation data on the vital sign spectrum data, thereby improving accuracy of the vital sign signal value.

In some embodiments, referring to FIG. 4, the environmental feature representation data of the environment where the measured object is located may be obtained through actions at steps S410 and S420.

At step S410, environmental data of the environment where the measured object is located is obtained based on a video signal. The video signal is obtained by capturing the environment where the measured object is located.

In an embodiment, the environment data of the environment where the measured object can be obtained based on the video signal captured by the video signal collection device. Exemplarily, the video signal may refer to a video image. The video image may be preprocessed. For example, preprocessing that can enhance image quality, such as denoising the video image and enhancing contrast of the video image, is performed. Further, an environment where the measured object is located in the preprocessed video image can be processed and analyzed to extract environmental data of the environment where the measured object is located in the video image. Exemplarily, the environmental data may include, for example, at least one of temperature, humidity, weather, wind speed, or light intensity.

Exemplarily, the video signal may carry at least one of pieces of information, for example, capture timestamp, capture location, latitude and longitude, or altitude. The environmental data of the environment where the measured object is located may be determined by extracting, based on the video image, at least one of the capture timestamp, the capture location, the latitude and longitude, or the altitude for estimation. The capture timestamp may include any one of spring, summer, autumn, and winter, any one of day and night, and any hour or any minute of 24 hours of the day. The capture location may be an indoor premise, an outdoor premise, or a semi-open premise. The semi-open premise may be a balcony or a terrace, a corridor or passageway, a rooftop or a roof deck, a greenhouse or a conservatory, etc.

At step S420, feature extraction is performed based on the environmental data in the video signal to obtain the environmental feature representation data. The environmental feature representation data includes at least one of a humidity feature, a temperature feature, a weather feature, a wind speed feature, or a light intensity feature.

In some cases, the environmental data may be a high-dimensional sparse feature vector. In order to facilitate subsequent prediction of the vital sign signal value of the measured object, embedding processing can be performed on the environmental data to generate a low-dimensional dense feature vector of a predetermined dimension to obtain the environmental feature representation data. As a result, a computational load and storage space can be reduced during the prediction of the vital sign signal value based on the environmental feature representation data, thereby improving the prediction efficiency.

For example, the environmental data may be represented as a high-dimensional sparse feature vector. The environmental feature representation data may refer to environmental feature representation data obtained by the environmental data subjected to the embedding. It can retain an important feature and information of the environmental data as the high-dimensional sparse feature vector while reducing dimensionality and complexity of the environmental data.

In the embodiment described above, the environmental data of the environment where the measured object is located is obtained based on the video signal, and the feature extraction is performed on the measured object based on the environmental data to obtain the environmental feature representation data. In this way, the environmental feature representation data of the environment where the measured object is located can be quickly obtained, which facilitates subsequent removal of the differential impact of the environmental feature representation data on the vital sign spectrum data, thereby improving the accuracy of the vital sign signal value.

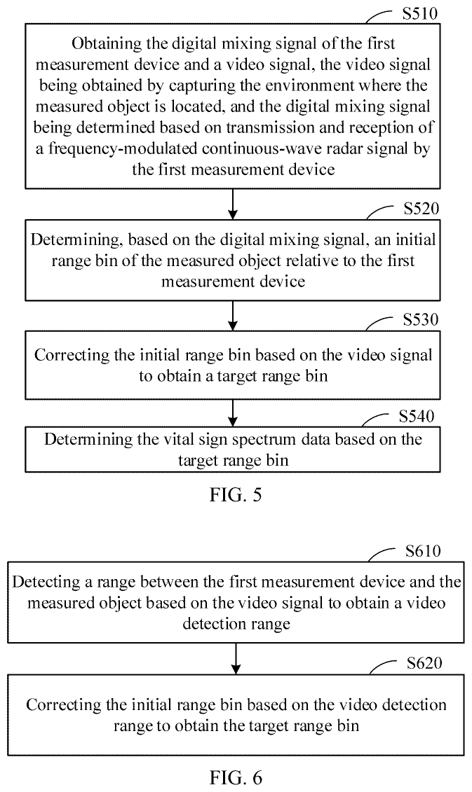

In some embodiments, referring to FIG. 5, the vital sign spectrum data of the measured object may be obtained through actions at steps S510 to S540.

At step S510, the digital mixing signal of the first measurement device and a video signal is obtained. The video signal is obtained by capturing the environment where the measured object is located, and the digital mixing signal is determined based on transmission and reception of a frequency-modulated continuous-wave radar signal by the first measurement device.

In some cases, the vital sign spectrum data of the measured object can be determined based on the digital mixing signal and the video signal. By determining the vital sign spectrum data based on the digital mixing signal combined with the video signal, the accuracy of the vital sign spectrum data can be improved.

In an embodiment, the environment where the measured object is located can be captured by the video signal collection device to obtain the video signal, and at the same time, the digital mixing signal determined based on the transmission and the reception of the frequency-modulated continuous-wave radar signal by the first measurement device using the millimeter wave radar is obtained. Thus, The vital sign spectrum data of the measured object is determined based on the digital mixing signal and the video signal.

Exemplarily, the video signal may have a capture timestamp, and the digital mixing signal may have corresponding time information. The capture timestamp of the video signal is consistent with the time information corresponding to the digital mixing signal, or the video signal and the digital mixing signal are time-aligned.

At step S520, an initial range bin of the measured object relative to the first measurement device is determined based on the digital mixing signal.

In an embodiment, the initial range bin may be determined based on a range determination (Range FFT) operation and a range bin determination (Range bin tracking) operation. Exemplarily, FFT may be performed on the digital mixing signal to determine a spectrum of the range dimension, to obtain a plurality of range bins. Then, a search is performed in the plurality of range bins to determine the range bin corresponding to the measured object as the initial range bin.

At step S530, the initial range bin is corrected based on the video signal to obtain a target range bin.

In an embodiment, the initial range bin can be corrected based on the video signal to obtain the target range bin corresponding to the measured object subsequent to the determining the initial range bin corresponding to the measured object. As a result, phase extracting can be performed based on the target range bin to determine a phase signal. Thus, the vital sign spectrum data of the measured object is determined based on the phase signal.

At step S540, the vital sign spectrum data is determined based on the target range bin.

Exemplarily, subsequent to the correcting the initial range bin as the target range bin based on the video signal, a phase extracting (Extract Phase) operation can be performed on the target range bin to obtain a phase corresponding to the measured object.

Exemplarily, the digital mixing signal may be determined by transmitting and receiving a chirp signal by the millimeter-wave radar. As an example, in a frame or in a frame period, a plurality of chirp signals may be continuously transmitted by a millimeter-wave radar. The frame period may refer to a complete transmission and reception period of a chirp signal. For example, when a frame period is 50 milliseconds and a duration of a chirp signal is 50 microseconds, 1,000 chirp signals are transmitted in a frame.

Exemplarily, the transmitted chirp signal and the received reflected chirp signal may be mixed to obtain a mixing signal, and then analog-to-digital conversion is performed on the mixing signal. When performing the analog-to-digital conversion, the mixing signal may be sampled based on the predetermined number of samples to obtain a digital mixing signal. The predetermined number of the samples is the number of samples in a chirp signal. The digital mixing signal may have a corresponding bandwidth.

As an example, for the digital mixing signal, the initial range bin is determined based on the range determination (Range FFT) operation and the range bin determination (Range bin tracking) operation. Then, the initial range bin is corrected based on the video signal to obtain the target range bin. Then, the target range bin is the phase extracting (Extract Phase) operation to determine the phase of the target range bin corresponding to the measured object. This can be performed cyclically. In this way, variation of the target range bin of the measured object over the number of the frames can be determined, that is, variation of the phase of the measured object over time can be determined. Phase unwrapping can be performed based on a phase unwrapping operation to obtain the phase signal of the measured object. Based on a phase differencing operation, the unwrapped phase signal can be enhanced, and an existing phase drift can be reduced. Based on a bandpass filtering operation, the corresponding phase in the phase signal can be filtered for differentiation, or a frequency formed by a corresponding phase change in the phase signal for differentiation, facilitating subsequent determination of different vital sign signals. The FFT can be performed on the obtained phase signal based on a spectrum estimation operation to obtain the vital sign spectrum data of the measured object.

Exemplarily, the vital sign spectrum data may be obtained by embedding on the result obtained based on the spectrum estimation operation. That is, the vital sign spectrum data is a low-dimensional dense feature vector with a predetermined dimension. Exemplarily, the vital sign spectrum data may at least include information on the number of transmitted waves, information on the number of transmitted wave samples, and bandwidth information. The information on the number of transmitted waves may be the number of chirp signals transmitted in a frame. The information on the number of transmitted wave samples may be the predetermined number of samples in a chirp signal. A bandwidth may refer to a bandwidth subjected to the bandpass filtering.

In the embodiment described above, the video signal is obtained by capturing the environment where the measured object is located. Meanwhile, the digital mixing signal is determined based on the transmission and the reception of the frequency-modulated continuous-wave radar signal by the first measurement device. Then, the initial range bin relative to the first measurement device is determined based on the digital mixing signal. The initial range bin is corrected based on the video signal to obtain the target range bin. The vital sign spectrum data is determined based on the target range bin. In this way, the correction of the initial range bin during the determination of the vital sign spectrum data of the measured object can be realized based on the video signal. Thus, the accuracy when measuring the vital sign signal of the measured object can be improved.

In some embodiments, referring to FIG. 6, the correcting the initial range bit based on the video signal to obtain the target range bit includes actions at steps S610 and S620.

At step S610, a range between the first measurement device and the measured object is detected based on the video signal to obtain a video detection range.

At step S620, the initial range bin is corrected based on the video detection range to obtain the target range bin.

In an embodiment, a determination can be made by comparing the video detection range and the initial range bin. If a difference between the video detection range and the initial range bin is greater than a predetermined difference threshold, the initial range bin can be corrected based on the video detection range. As an example, the expression “the initial range bin is corrected based on the video detection range” may refer to researching being performed among a plurality of range bins obtained based on a spectrum of the range dimension when the video detection range is greater than the predetermined difference threshold to determine the range bin corresponding to the measured object as the target range bin. As another example, the expression “the initial range bin is corrected based on the video detection range” may refer to the initial range bin being adjusted based on the video detection range when the video detection range is greater than the predetermined difference threshold to obtain the target range bin.

In the embodiment described above, the video detection range between the first measurement device and the measured object can be determined based on the video signal, and the initial range bin during the determination of the vital sign spectrum data of the measured object can be corrected based on the video detection range. Thus, the accuracy when measuring the vital sign signal of the measured object can be improved.

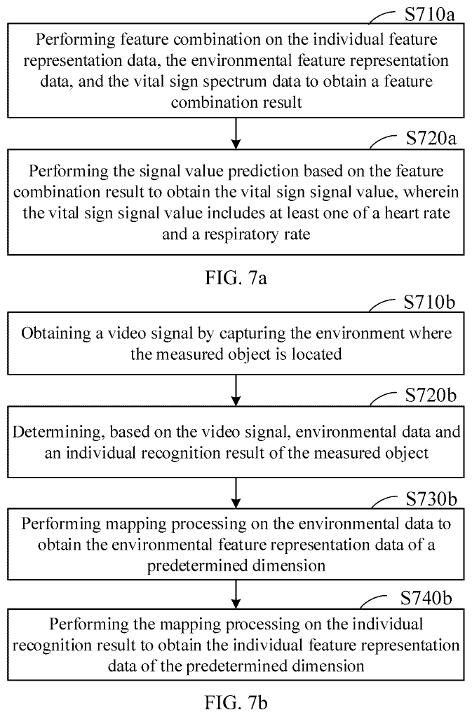

In some embodiments, referring to FIG. 7a, performing the signal value prediction based on the individual feature representation data, the environmental feature representation data, and the vital sign spectrum data to obtain the vital sign signal value with the differential impact removed may include actions at steps S710a to S720a.

At step S710a, feature combination is performed on the individual feature representation data, the environmental feature representation data, and the vital sign spectrum data to obtain a feature combination result.

In an embodiment, the feature combination can be performed on the individual feature representation data, the environmental feature representation data, and the vital sign spectrum data to allow the individual feature representation data, the environmental feature representation data, and the vital sign spectrum data to be converted into a feature combination result suitable for model training. Exemplarily, if each of the individual feature representation data, the environmental feature representation data, and the vital sign spectrum data is the low-dimensional dense feature vector with the predetermined dimension, vector concatenation is performed on the individual feature representation data, the environmental feature representation data, and the vital sign spectrum data to obtain a concatenated vector as the feature combination result.

At step S720a, the signal value prediction is performed based on the feature combination result to obtain the vital sign signal value. The vital sign signal value includes at least one of a heart rate and a respiratory rate.

Exemplarily, the signal value prediction can be performed based on the feature combination result to obtain the heart rate. The signal value prediction can be performed based on the feature combination result to obtain the respiratory rate. In addition, the signal value prediction can be performed based on the feature combination result to obtain the heart rate and the respiratory rate.

As an example, phases in phase signals corresponding to the heart rate and the respiratory rate are typically different, or frequencies of phase changes in the phase signals corresponding to the heart rate and the respiratory rate are typically different. In other words, bandwidth information in the vital sign spectrum data corresponding to the heart rate and the respiratory rate is different. In this way, the heart rate or the respiratory rate can be obtained by performing the signal value prediction based on the vital sign spectrum data subjected to the bandpass filtering.

Exemplarily, the signal value prediction may be performed based on different confidence metrics.

In the embodiment described above, the feature combination is performed on the individual feature representation data, the environmental feature representation data, and the vital sign spectrum data to obtain the feature combination result, and the signal value prediction is performed based on the feature combination result to obtain the vital sign signal value including at least one of a heart rate and a respiratory rate. In this way, the differential impact of each of the individual feature representation data and the environmental feature representation data on the vital sign spectrum data can be removed. As a result, the accuracy when measuring the vital sign signal of the measured object can be improved.

In some embodiments, the performing the feature combination on the individual feature representation data, the environmental feature representation data, and the vital sign spectrum data to obtain the feature combination result may include: performing concatenation processing on the individual feature representation data, the environmental feature representation data, and the vital sign spectrum data to obtain the feature combination result.

In an embodiment, each of the individual feature representation data, the environmental feature representation data, and the vital sign spectrum data may be a predetermined dimension. The individual feature representation data, the environmental feature representation data, and the vital sign spectrum data can be concatenated based on the predetermined dimension when performing the feature combination on the individual feature representation data, the environmental feature representation data, and the vital sign spectrum data, to obtain the feature combination result, allowing the vital sign signal values to be predicted based on the feature combination result.

In the embodiment described above, by concatenating the individual feature representation data and the environmental feature representation data to the vital sign spectrum data, the differential impact of each of the individual feature representation data and the environmental feature representation data on the vital sign spectrum data is taken into account and removed when measuring the vital sign signal of the measured object. Thus, the accuracy of measuring the vital sign signal can be improved.

In some embodiments, referring to FIG. 7b, the obtaining the individual feature representation data of the measured object and the environmental feature representation data of the environment where the measured object is located may include actions at steps S710b to S740b.

At S710b, a video signal is obtained by capturing the environment where the measured object is located.

At S720b, environmental data and an individual recognition result of the measured object are determined based on the video signal.

At S730b, mapping processing is performed on the environmental data to obtain the environmental feature representation data of a predetermined dimension.

At S740b, the mapping processing is performed on the individual recognition result to obtain the individual feature representation data of the predetermined dimension.

In an embodiment, the video signal captured by the video signal collection device for the environment where the measured object is located can be obtained, and the biometric feature data of the measured object can be obtained based on the video signal. Exemplarily, the obtained biometric feature data may be facial data of the measured object. For example, a facial region of the measured object can be recognized based on the video signal. When the facial region is detected, facial features such as a facial contour, eyes, a mouth, and a nose can be further extracted to obtain facial data. Individual recognition can be performed based on the facial data to obtain the individual recognition result of the measured object including at least one of identity identification, a gender feature, an age feature, or a skin type feature. Then, the mapping processing can be performed on the individual recognition result to obtain the individual feature representation data of the predetermined dimension.

In an embodiment, the video signal captured by the video signal collection device for the environment where the measured object is located can be obtained, and the environmental data of the environment where the measured object is located can be obtained based on the video signal. Exemplarily, the obtained environmental data may include, for example, at least one of temperature, humidity, weather, wind speed, or light intensity. Exemplarily, the video signal may have at least one piece of information, for example, capture timestamp, capture location, latitude and longitude, or altitude. Exemplarily, the environmental data of the environment where the measured object is located is determined based on the at least one piece of information, for example, capture timestamp, capture location, latitude and longitude, or altitude. Then, mapping processing is performed on the environmental data to obtain the environmental feature representation data of the predetermined dimension.

The predetermined dimension of the environmental feature representation data is the same as the predetermined dimension of the individual feature representation data.

Exemplarily, the vital sign spectrum data may be in a predetermined dimension. When the environmental data and the individual recognition result are respectively mapped into the environmental feature representation data and the individual feature representation data of the predetermined dimension, the predetermined dimension can be determined based on the predetermined dimension of the vital sign spectrum data.

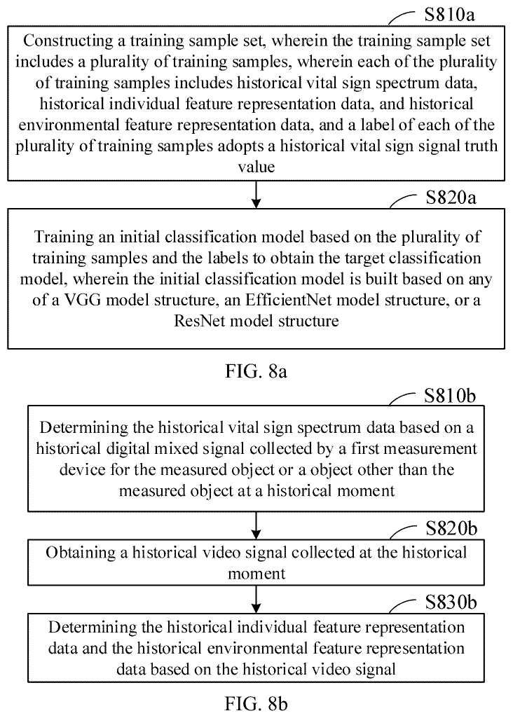

In some embodiments, the vital sign signal value may be outputted by a target classification model. Referring to FIG. 8a, the target classification model can be obtained by the following training method.

At S810a, a training sample set is constructed. The training sample set includes a plurality of training samples. Each of the plurality of training samples includes historical vital sign spectrum data, historical individual feature representation data, and historical environmental feature representation data, and a label of each of the plurality of training samples adopts a historical vital sign signal truth value.

At S820a, an initial classification model is trained based on the plurality of training samples and the labels to obtain the target classification model. The initial classification model is built based on any of a VGG model structure, an EfficientNet model structure, or a ResNet model structure.

In an embodiment, the historical individual feature representation data, the historical environmental feature representation data, and the historical vital sign spectrum data in the training sample set can be inputted into the initial classification model after constructing the training sample set, and feature combination can be performed on the historical individual feature representation data, the historical environmental feature representation data, and the historical vital sign spectrum data through the initial classification model to obtain a historical feature combination result. Prediction is made based on the historical feature combination result to obtain a predicted vital sign signal. Further, the initial classification model corresponds to a loss function, and an input of the initial classification model corresponds to a label. The label and the predicted vital sign signal are inputted into the loss function to determine a model loss value, and the parameter of the initial classification model is updated based on the determined model loss value. This loop repeats until a model training stop condition is met. In this way, the target classification model is obtained. The model training stop condition may be that the model loss value tends to converge, or the number of training epochs reaches a predetermined number of epochs.

In the embodiment described above, the training samples are constructed using the historical vital sign spectrum data, the historical individual feature representation data, and the historical environmental feature representation data. During the training of the initial classification model using the training samples and the historical vital sign signal truth value, the initial classification model gradually acquires an ability to removing the differential impact of each of the individual feature representation data and the environmental feature representation data on the vital sign spectrum data. In this way, the target classification model is obtained. Thus, accurate vital sign signal value can be obtained.

In some embodiments, referring to FIG. 8b, the training samples may be constructed by the following processes.

At S810b, the historical vital sign spectrum data is determined based on a historical digital mixing signal collected by a first measurement device for the measured object or an object other than the measured object at a historical moment.

At S820b, a historical video signal collected at the historical moment is obtained.

At S830b, the historical individual feature representation data and the historical environmental feature representation data are obtained based on the historical video signal.

In an embodiment, since the model training requires the training samples, at a plurality of historical moments before the model training, the historical digital mixing signal determined through transmission and reception of the frequency-modulated continuous-wave radar signal by the first measurement device using the millimeter-wave radar is obtained, and the historical initial range bin of the measured object or the object other than the measured object relative to the first measurement device is determined based on the historical digital mixing signal. Then, a range between the first measurement device and the measured object or the object other than the measured object is detected based on the historical video signal to obtain a historical video detection range. The historical initial range bin is corrected based on the historical video detection range to obtain a historical target range bin. The historical vital sign spectrum data is determined based on the historical target range bin.

In an embodiment, the biometric feature data of the measured object or biometric feature data of the object other than the measured object can be obtained based on the historical video signal, and individual recognition can be performed on the measured object or the object other than the measured object based on the biometric feature data in the historical video signal to obtain the historical individual feature representation data.

In an embodiment, the environmental data of the environment where the measured object is located or environmental data of an environment where the object other than the measured object is located can be obtained based on the historical video signal, and feature extraction can be performed based on the environmental data in the historical video signal to obtain the historical environmental feature representation data.

In an embodiment, the historical vital sign signal truth value can also be collected and determined by the second measurement device for the measured object or the object other than the measured object to take the historical vital sign signal truth value as a label for the model training.

At this point, training samples inputted into the initial classification model can be constructed based on the historical vital sign spectrum data, the historical individual feature representation data, and the historical environmental feature representation data.

It should be noted that the historical vital sign spectrum data, the historical individual feature representation data, the historical environmental feature representation data, and the historical vital sign signal truth value are time-aligned. That is, each of the historical vital sign spectrum data, the historical individual feature representation data, the historical environmental feature representation data, and the historical vital sign signal truth value is determined at one historical moment among the plurality of historical moments.

In the embodiment described above, the training samples are constructed using the historical vital sign spectrum data, the historical individual feature representation data, and the historical environmental feature representation data to provide a data basis for training the initial classification model.

Embodiments of the present disclosure provide a human factor intelligence-based vital sign signal measurement method, which may be applied in a first measurement device or a data processing device in a vital sign signal measurement system. The vital sign signal measurement method may include actions at steps S901 to S915.

At step S901, historical vital sign spectrum data is obtained. The historical vital sign spectrum data is collected and determined by a first measurement device for a measured object or an object other than the measured object at a historical moment.

At step S903, a historical video signal collected at the historical moment is obtained.

At step S905, a historical vital sign signal truth value is obtained. The historical vital sign signal truth value is collected and determined by a second measurement device for the measured object or the object other than the measured object at the historical moment.

At step S907, historical individual feature representation data and historical environmental feature representation data are obtained based on the historical video signal.

At step S909, a training sample is constructed based on the historical individual feature representation data, the historical environmental feature representation data, and the historical vital sign spectrum data.

It should be noted that a plurality of training samples form a training sample set. Each of the plurality of training samples includes the historical vital sign spectrum data, the historical individual feature representation data, and the historical environmental feature representation data, and a label of each of the plurality of training samples adopts a historical vital sign signal truth value.

At step S911, an initial classification model is trained based on the plurality of training samples and the labels to obtain the target classification model.

At step S913, individual feature representation data of a measured object, environmental feature representation data of an environment where the measured object is located, and vital sign spectrum data of the measured object are obtained. The individual feature representation data and the environmental feature representation data have a differential impact on the vital sign spectrum data.

In an embodiment, the obtaining the individual feature representation data of the measured object includes: obtaining biometric feature data of the measured object based on a video signal, wherein the video signal is obtained by capturing the environment where the measured object is located; and performing individual recognition on the measured object based on the biometric feature data in the video signal to obtain the individual feature representation data.

In an embodiment, the biometric feature data of the measured object is any one of facial data, iris data, retinal data, or eyeprint data.

In an embodiment, the individual feature representation data includes at least one of a gender feature, an age feature, or a skin type feature of the measured object.

In an embodiment, the obtaining the environmental feature representation data of the environment where the measured object is located includes: obtaining, based on a video signal, environmental data of the environment where the measured object is located, wherein the video signal is obtained by capturing the environment where the measured object is located; and performing feature extraction based on the environmental data in the video signal to obtain the environmental feature representation data, wherein the environmental feature representation data includes at least one of a humidity feature, a temperature feature, a weather feature, or a wind speed feature.

In an embodiment, the obtaining the vital sign spectrum data of the measured object includes: obtaining the digital mixing signal of the first measurement device and a video signal, wherein the video signal is obtained by capturing the environment where the measured object is located, and the digital mixing signal is determined based on transmission and reception of a frequency-modulated continuous-wave radar signal by the first measurement device; determining, based on the digital mixing signal, an initial range bit of the measured object relative to the first measurement device; correcting the initial range bit based on the video signal to obtain a target range bit; and determining the vital sign spectrum data based on the target range bit.

In an embodiment, the correcting the initial range bit based on the video signal to obtain the target range bit includes: detecting a range between the first measurement device and the measured object based on the video signal to obtain a video detection range; and correcting the initial range bit based on the video detection range to obtain the target range bit.

At step S915, signal value prediction is performed based on the individual feature representation data, the environmental feature representation data, and the vital sign spectrum data to obtain a vital sign signal value with the differential impact removed.

In an embodiment, the vital sign signal value is outputted by a target classification model.

In an embodiment, feature combination is performed on the individual feature representation data, the environmental feature representation data, and the vital sign spectrum data to obtain a feature combination result; and the signal value prediction is performed based on the feature combination result to obtain the vital sign signal value, wherein the vital sign signal value includes at least one of a heart rate and a respiratory rate.

It can be understood that in the various embodiments of the present disclosure, numerical values of sequence numbers of the above processes do not mean an execution order and should not constitute any limitation to an implementation process of the embodiments of the present disclosure as the execution order of individual processes should be determined by their functions and internal logics.

Embodiments of the present disclosure provide a human factor intelligence-based vital sign signal measurement apparatus. The vital sign signal measurement apparatus may be applied in a first measurement device or a data processing device in a vital sign signal measurement system. The measurement device may include a feature data obtaining module and a vital sign determination module.

The feature data obtaining module is configured to obtain individual feature representation data of a measured object, environmental feature representation data of an environment where the measured object is located, and vital sign spectrum data of the measured object. The individual feature representation data and the environmental feature representation data have a differential impact on the vital sign spectrum data.

The vital sign determination module is configured to perform a signal value prediction based on the individual feature representation data, the environmental feature representation data, and the vital sign spectrum data to obtain a vital sign signal value with the differential impact removed.

In some embodiments, the feature data obtaining module is further configured to: obtain biometric feature data of the measured object based on a video signal, wherein the video signal is obtained by capturing the environment where the measured object is located; and perform individual recognition on the measured object based on the biometric feature data in the video signal to obtain the individual feature representation data. The biometric feature data of the measured object is any one of facial data, iris data, retinal data, or eyeprint data; and the individual feature representation data includes at least one of a gender feature, an age feature, or a skin type feature of the measured object.

In some embodiments, the feature data obtaining module is further configured to: obtain, based on a video signal, environmental data of the environment where the measured object is located, wherein the video signal is obtained by capturing the environment where the measured object is located; and perform feature extraction based on the environmental data in the video signal to obtain the environmental feature representation data, wherein the environmental feature representation data includes at least one of a humidity feature, a temperature feature, a weather feature, or a wind speed feature.

In some embodiments, the feature data obtaining module is further configured to: obtain the digital mixing signal of the first measurement device and a video signal, wherein the video signal is obtained by capturing the environment where the measured object is located, and the digital mixing signal is determined based on transmission and reception of a frequency-modulated continuous-wave radar signal by the first measurement device through a millimeter wave radar; determine, based on the digital mixing signal, an initial range bit of the measured object relative to the first measurement device; correct the initial range bit based on the video signal to obtain a target range bit; and determine the vital sign spectrum data based on the target range bit.

In some embodiments, the feature data obtaining module is further configured to: detect a range between the first measurement device and the measured object based on the video signal to obtain a video detection range; and correct the initial range bit based on the video detection range to obtain the target range bit.

In some embodiments, the vital sign determination module is further configured to: perform feature combination on the individual feature representation data, the environmental feature representation data, and the vital sign spectrum data to obtain a feature combination result; and perform the signal value prediction based on the feature combination result to obtain the vital sign signal value, wherein the vital sign signal value includes at least one of a heart rate and a respiratory rate.

In some embodiments, the vital sign determination module is further configured to perform concatenation processing on the individual feature representation data, the environmental feature representation data, and the vital sign spectrum data to obtain the feature combination result.

In some embodiments, the feature data obtaining module is further configured to: obtain a video signal obtained by capturing the environment where the measured object is located; determine, based on the video signal, environmental data and an individual recognition result of the measured object; perform mapping processing on the environmental data to obtain the environmental feature representation data of a predetermined dimension; and perform the mapping processing on the individual recognition result to obtain the individual feature representation data of the predetermined dimension.

In some embodiments, the vital sign signal value is outputted by a target classification model. The measurement apparatus may further include a model training module. The model training module is configured to: construct a training sample set, wherein the training sample set includes a plurality of training samples, each of the plurality of training samples includes historical vital sign spectrum data, historical individual feature representation data, and historical environmental feature representation data, and a label of each of the plurality of training samples adopts a historical vital sign signal truth value; and train an initial classification model based on the plurality of training samples and the labels to obtain the target classification model, wherein the initial classification model is built based on any one of a VGG model structure, an EfficientNet model structure, or a ResNet model structure.

In some embodiments, the model training module is further configured to construct the training samples by: determining the historical vital sign spectrum data based on a historical digital mixing signal collected by a first measurement device for the measured object or an object other than the measured object at a historical moment; obtaining a historical video signal collected at the historical moment; and determining the historical individual feature representation data and the historical environmental feature representation data based on the historical video signal.

For the specific functions and effects of the measurement apparatus, reference can be made to other embodiments of the present disclosure, and details thereof are omitted herein. The modules of the measurement apparatus may be fully or partly implemented through software, hardware, and a combination thereof. The modules may be embedded or independent of a processor of a computer device in the form of hardware or stored in a memory of a computer device in the form of software, and thus the processor can invoke and perform the above operations corresponding to the modules.

Embodiments of the present disclosure also provide a computer device. The computer device includes a memory and a processor. The memory has a computer program stored thereon. The processor is configured to implement, when executing the computer program, the vital sign signal measurement method in the embodiments described above.

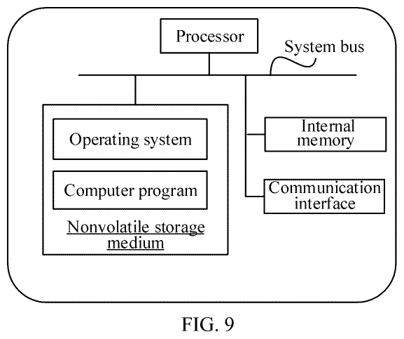

In this embodiment, referring to FIG. 9, the computer device may be a terminal, and its internal structural diagram may be as shown in FIG. 9. The computer device includes a processor, a memory, and a communication interface that are connected via a system bus. The processor of the computer device is configured to provide computing and control capabilities. The memory of the computer device includes a nonvolatile storage medium and an internal memory. The nonvolatile storage medium is stored with an operating system and a computer program. The internal memory provides an environment for operation of the operating system and the computer program in the nonvolatile storage medium. The communication interface of the computer device is configured to be in communication with an external terminal in a wired or wireless manner. The wireless manner can be implemented through WIFI, an operator network, NFC (near field communication), or other technologies. The computer program is configured to implement, when executed by the processor, the vital sign signal measurement method in any one of the embodiments describe above.

Embodiments of the present disclosure also provide an edge computing device. The edge computing device includes a memory, a processor, and a communication interface. The memory has a computer program stored thereon. The processor is configured to implement, when executing the computer program, the vital sign signal measurement method in any one of the embodiments described above.

### Embodiment 2

In order to solve problems that a signal is affected by an environment when using a millimeter-wave radar for vital sign detection, and there are significant differences in reflected signals between different individuals, embodiments of the present disclosure combine the millimeter-wave radar with remote photoplethysmography (rPPG), thereby enabling more accurate vital sign detection. An execution process of a human factor intelligence-based vital sign signal detection method according to embodiments of the present disclosure may include actions at steps 1 to 4.

At step 1, a radio-frequency reflection signal about a target object and continuous multi-frame image data are obtained. A detection principle of the millimeter-wave radar is to detect a phase change within a specific range caused by a tiny vibration of a target. A principle of the rPPG is that blood flow caused by heart beating causes a subtle brightness change in a skin, and reflected surrounding light is used to measure the subtle brightness change in the skin, thereby realizing detection of a vital sign. In the embodiments of the present disclosure, more accurate vital sign signal is obtained by combining the millimeter-wave radar with the rPPG technology. That is, the vital sign signal is generated by obtaining a radio-frequency reflection signal required for the detection by the millimeter-wave radar and continuous multi-frame image data (i.e., video data) required for the rPPG.

The above radio-frequency reflection signal can be obtained by transmitting a target pulse signal to the target object through the millimeter-wave radar and collecting an initial reflected signal after the pulse signal is reflected by the target object. The obtained initial reflected signal is then preprocessed to obtain the radio-frequency reflection signal. The preprocessing may include noise reduction, filtering, etc. For the continuous multi-frame image data, it can be collected by a video recording device. The radio-frequency reflection signal and the continuous multi-frame image data are cropped to obtain the required radio-frequency reflection signal and multi-frame image data. For example, a total of 1 minute of radio-frequency reflection signals and 1 minute of multi-frame image data are collected. Moreover, a time point of starting collecting the radio-frequency reflection signals is consistent with a time point of starting collecting the multi-frame image data. When it is necessary to obtain 30 seconds of data to generate vital sign signals, since the time point of starting collecting the radio-frequency reflection signal is the same as the time point of starting collecting the multi-frame image data, the collected 1-minute radio-frequency reflection signal can be cropped to retain the a first 30 seconds of radio-frequency reflection signals. Similarly, for the multi-frame image data, a first 30 seconds of multi-frame image data are retained. Then, radio-frequency reflection signals and multi-frame image data with the same collection start time point and collection end time point are obtained.

At step 2, first feature data is obtained based on the radio-frequency reflection signal, and second feature data is obtained based on the multi-frame image data. In an embodiment, for the first feature data, a range curve can be extracted from the collected radio-frequency reflection signals. The method adopted may be performing FFT on the collected radio-frequency reflection signals. Then, a plurality of range bins in the range curve are determined. A target pulse signal transmitted to the target object by the millimeter-wave radar is denoted as s(t), and a target pulse signal received at a receiving terminal is denoted as r(t). Then, a mixed and filtered intermediate frequency signal received on an object at a range of R is denoted as R(t). The radio-frequency reflection signal at step 1 is the intermediate-frequency signal R(t). For objects at different ranges, their reflected signals have different frequencies. Therefore, the FFT is performed on the intermediate-frequency signal, and a duration of the millimeter-wave radar is divided into different range intervals, which are referred to as range bins. In order to measure a small-scale change of the target object, it is necessary to measure a phase change of the intermediate-frequency signal R(t) within the range bin. Therefore, after determining a plurality of range bins, it is necessary to determine phases at the plurality of range bins to construct a phase signal based on the phases at the plurality of range bins. The first feature data is obtained based on the phase signal. A vibration signal, i.e., a heartbeat signal or a respiratory signal, is obtained by continuously extracting a phase at the range bin m within a predetermined time period. The first feature data may be implemented in the form of a spectrum estimation result of the vibration signal. The first feature data may be specifically implemented as a matrix form of K×M×N. K represents the number of transmitted waves in a frame (if a frame period is 50 ms and Tc is 50 us, K is 1,000), M represents the number of samples in a pulse, and N represents a bandwidth (depending on characteristics of a heart rate and a respiratory rate, a bandwidth of the heart rate differs from a bandwidth of the respiratory rate).

For the second feature data, it can be extracted by cropping image data in a region of interest (ROI). In an embodiment, a region of interest in the multi-frame image data can be determined, which includes a facial region, etc. The region of interest can be determined through an object detection algorithm (such as a facial detection algorithm). Then, the multi-frame image data is cropped based on the determined region of interest to obtain a corresponding plurality of pieces of region-of-interest image data. Then, the plurality of pieces of region-of-interest image data are converted from the RGB-encoded data into YUV-encoded data to obtain a corresponding plurality of pieces of region-of-interest chromaticity data. Finally, the second feature data is obtained based on the plurality of pieces of region-of-interest chromaticity data. In an embodiment, a remote photoplethysmography signal can be extracted from the plurality of pieces of region-of-interest chromaticity data. Thus, the second feature data is obtained based on the remote photoplethysmography signal.

At step 3, feature fusion is performed on the first feature data and the second feature data to obtain a fused feature. The feature fusion may be implemented as feature-level fusion, weighted fusion, feature concatenation, feature stacking, feature selection, feature crossing, etc.

At step 4, vital sign signal of the target object is obtained based on the fused feature. In an embodiment, a neural network model can be used to obtain the vital sign signal. In an embodiment, the fused feature can be inputted into a pretrained target neural network model. Then, the vital sign signal outputted by the target neural network model is obtained.

In some embodiments, the target neural network model may be implemented as a vision transformer network model (a ViT network model for short). A method for training the target neural network model may include: detecting a to-be-detected target to obtain a radio-frequency reflection signal about the to-be-detected target, continuous multi-frame image data, and vital sign signal. The vital sign signal can be obtained by collecting the to-be-detected target by a vital sign collection device (such as a medical instrument). Then, signal alignment between the radio-frequency reflection signal and the vital sign signal is performed to obtain a vital sign true value signal, and feature fusion is performed on the radio-frequency reflection signal and the multi-frame image data to obtain the vital sign feature signal. A feature of the radio-frequency reflection signal and a feature of the multi-frame image data can be extracted separately before fusion and undergo the feature fusion. Specific steps thereof are the same as the above steps of performing the feature fusion on the first feature data and the second feature data to obtain the fused feature, and details thereof are omitted herein.

Then, a training data set is constructed based on the vital sign feature signal and the vital sign truth value signal, and iterative training is performed on the initial model based on the training data set. The training is expired in response to detecting that the initial model meets a predetermined condition, to obtain the target neural network model. The predetermined condition may be set as model convergence or an accuracy rate reaching a predetermined threshold.

In a specific embodiment, the human factor intelligence-based vital sign signal detection method according to the embodiments of the present disclosure mainly involves three steps. The first two steps are in no order of priority, which includes: obtaining a millimeter-wave radar signal feature and obtaining a rPPG signal feature. The third step is to input the two features obtained above into the trained ViT network model to obtain the outputted vital sign signal.

In the step of obtaining the millimeter-wave radar signal feature, its specific implementation includes the following steps.
(1) Range FFT: The FFT is performed on the collected radio-frequency reflection signal to obtain a range curve.
(2) Range bin tracking: A range interval of a target can be determined through an approximate positional relationship between the radar and a human body, and a maximum value within this interval can be searched to obtain a range bin corresponding to the target.
(3) Extract Phase: A phase is extracted at the range bin of the target.

Then, the steps (1) to (3) are performed in a loop. A frame period is 50 ms, that is, a phase of the target is extracted once in each frame period. If a radial range between the target and the range changes, a rang bin at this point is obtained based on the range bin tracking algorithm. Then, the phase is extracted, and variation of the phase of the target over the number of frames is obtained by transmitting N frames cyclically, which can also be regarded as a relationship between the phase of the target and time, denoted as a vibration signal x(t).
(4) Phase Unwrapping: Since a phase value is within [−π,π], it needs to be unwrapped to obtain an actual displacement curve. Therefore, whenever a phase difference between consecutive values is greater than/less than +π, the phase unwrapping is performed by subtracting 2π from the phase.
(5) Phase Difference: A phase difference operation is performed on the unwrapped phase by subtracting the consecutive phase values. This helps enhance the heartbeat signal and eliminates any phase drift.
(6) Bandpass Filtering: In accordance with a difference between a heartbeat frequency and a respiratory frequency, the phase values are filtered by using a bandpass filter for differentiation.
(7) Spectral Estimation: For the respiratory frequency, the FFT can be performed on the above phase signal distinguished as belonging to the respiratory frequency, and corresponding respiratory frequencies in an N Frame durations can be obtained based on peak magnitude and its harmonic characteristic. A respiratory frequency over a period of time is recorded, the respiratory frequency at this time is determined based on different confidence indicators, and a relationship between the respiratory frequency and time is outputted. For the heart rate, the above phase signal distinguished as belonging to the heart rate can be filtered first, aiming at reducing an impact of a relative positional movement of the human body on the measurement of the heart rate. (Because the measurement of the heart rate is based on a phase change caused by a range difference generated by a slight movement of cardiac contraction and relaxation. According to a micro-doppler principle, when a body of an individual swings sharply, it affects its accuracy.) By segmenting the samples herein, a threshold is set to determine whether it falls within a variation range of the heart rate, and data in a stable state is selected to perform the spectral estimation. That is, the FFT can be performed on the filtered phase signal belonging to the heart frequency, and corresponding heart frequencies in an N Frame durations are obtained based on peak magnitude and its harmonic characteristic. A heart frequency over a period of time is recorded, the heart frequency at this time is determined based on different confidence indicators, and a relationship between the heart frequency and time is outputted. An autocorrelation mechanism can be introduced during the recording to improve accuracy of the output.

Since then, the respiratory rate over time and the heart rate over time have been obtained. After that, the model's perception of the feature can be strengthened by using long-sequence supervised information. The structure adopted may be an encoder-decoder structure, in which the millimeter-wave radar signal feature is obtained from the encoder. The structure of each of the encoder and the decoder can adopt 1 stm, and an output of encoder is the millimeter-wave radar signal feature. The millimeter-wave radar signal feature is expressed as a K*M*N matrix, where K represents the number of transmitted waves in one frame; M represents the number of samples in a chirp; and N represents a bandwidth.

For the obtaining of rPPG signal feature, facial recognition is required to obtain an ROI of a face. The ROI is a multi-frame facial image (an RGB image). Then, based on the multi-frame face image, a YUV image that better characterizes brightness information is generated. The YUV image includes data from 6 channels, i.e., R channel data, G channel data, B channel data, Y channel data, U channel data, and V channel data. A CHROM signal, a POS (Plane-Othogonal-to-Skin) signal, and a filtered signal are extracted from each of the 6 channels. The specific implementation process of this step is as follows. The face is aligned cross different frames (i.e., the multi-frame face image data) based on the detected landmarks, and then the facial region is divided into n ROI blocks, R1, R2, . . . , Rn. An average color value is calculated for each color channel in each block. Average color values per channel of a same block position across different frames are concatenated into sequences, i.e., R1, G1, B1, R2, G2, B2, . . . , Rn, Gn, Bn. The sequences from the same color channel are connected to mappings (R, G, and B) of size N×L, where N=n. In addition, RGB color space is converted to YUV color space (Y, U, and V). Then, the CHROM algorithm and the POS algorithm are used for processing to correspondingly obtain the CHROM signal and the POS signal. Moreover, filtering is performed through Butterworth bandpass filter or other filters to obtain the filtered signal. Finally, different combined signals are concatenated into four spatio-temporal maps (STMaps) as the rPPG signal feature. The four STMaps include CHROM-STMap, POS-STMap, Filtered-STMap, and Original-STMap. The matrix of STMap is represented as B*S*T, where B represents a frame rate, S represents the number of ROI blocks, and T represents a time length.

The specific implementation process of the above third step is as follows. The millimeter-wave radar signal feature of K*M*N and the rPPG signal feature of B*S*T are fused. The fusion can adopt flattening to one-dimensional signal and passing through a fully connected layer to a fixed dimension (such as 1,024), and then the millimeter-wave radar signal feature is converted into a 1,024-dimensional vector. The rPPG signal feature is transformed into 4 1,024-dimensional vectors. There are 5 1,024-dimensional vectors through concatenation as the input to the VIT neural network model. Then, the final vital sign signal is outputted by the ViT neural network model.

Corresponding to the above human factor intelligence-based vital sign signal detection method, embodiments of the present disclosure provide a human factor intelligence-based vital sign signal detection apparatus. The apparatus includes an obtaining module, a feature extraction module, a feature fusion module, and a processing module.

The obtaining module is configured to obtain a radio-frequency reflection signal about a target object and continuous multi-frame image data.

A feature extraction module is configured to: obtain first feature data based on the radio-frequency reflection signal; and obtain second feature data based on the multi-frame image data.

The feature fusion module is configured to perform feature fusion on the first feature data and the second feature data to obtain a fused feature.

The processing module is configured to obtain vital sign signal of a target object based on the fused feature.

The human factor intelligence-based vital sign signal detection apparatus can be used to perform the technical solutions of the method embodiments described above, and for the implementation principles and technical effects thereof, reference can be further made to the relevant description in the method embodiments.

Corresponding to the human factor intelligence-based vital sign signal detection describe above, embodiments of the present disclosure provide an electronic device. Components of the electronic device may include, but are not limited to, one or more processors, a communication interface, and a memory, and a communication bus connecting different system components (including the memory, the communication interface, and a processor). For details, reference can be made to the relevant contents of the computer device shown in FIG. 9 in Embodiment 1.

Embodiments of the present disclosure also provide a computer-readable storage medium. The computer-readable storage medium has a computer program stored thereon. The computer program is configured to perform, when executed by a computer, the above vital sign signal measurement method in Embodiment 1 or the above human factor intelligence-based vital sign signal detection method in Embodiment 2.

Embodiments of the present disclosure also provide a computer program product including instructions. The instructions, when executed by a computer, cause the computer to perform the above vital sign signal measurement method in Embodiment 1 or the above human factor intelligence-based vital sign signal detection method in Embodiment 2.

What is claimed is:

1. A human factor intelligence-based vital sign signal measurement method, comprising:
obtaining individual feature representation data of a measured object, environmental feature representation data of an environment where the measured object is located, and vital sign spectrum data of the measured object, wherein the individual feature representation data and the environmental feature representation data have a differential impact on the vital sign spectrum data; and
performing signal value prediction based on the individual feature representation data, the environmental feature representation data, and the vital sign spectrum data to obtain a vital sign signal value with the differential impact removed.

2. The measurement method according to claim 1, wherein said obtaining the individual feature representation data of the measured object comprises:
obtaining biometric feature data of the measured object based on a video signal, the video signal being obtained by capturing the environment where the measured object is located; and
performing individual recognition on the measured object based on the biometric feature data in the video signal to obtain the individual feature representation data.

3. The measurement method according to claim 2, wherein:
the biometric feature data of the measured object is any one of facial data, iris data, retinal data, or eyeprint data; and
the individual feature representation data comprises at least one of a gender feature, an age feature, or a skin type feature of the measured object.

4. The measurement method according to claim 1, wherein said obtaining the environmental feature representation data of the environment where the measured object is located comprises:
obtaining, based on a video signal, environmental data of the environment where the measured object is located, the video signal being obtained by capturing the environment where the measured object is located; and
performing feature extraction based on the environmental data in the video signal to obtain the environmental feature representation data, the environmental feature representation data comprising at least one of a humidity feature, a temperature feature, a weather feature, or a wind speed feature.

5. The measurement method according to claim 1, wherein the vital sign spectrum data of the measured object is obtained based on a digital mixing signal of a first measurement device.

6. The measurement method according to claim 5, wherein said obtaining the vital sign spectrum data of the measured object based on the digital mixing signal of the first measurement device comprises:
obtaining the digital mixing signal of the first measurement device and a video signal, the video signal being obtained by capturing the environment where the measured object is located, and the digital mixing signal being determined based on transmission and reception of a frequency-modulated continuous-wave radar signal by the first measurement device through a millimeter wave radar;
determining, based on the digital mixing signal, an initial range bin of the measured object relative to the first measurement device;
correcting the initial range bin based on the video signal to obtain a target range bin; and
determining the vital sign spectrum data based on the target range bin.

7. The measurement method according to claim 6, wherein said correcting the initial range bin based on the video signal to obtain the target range bin comprises:
detecting a range between the first measurement device and the measured object based on the video signal to obtain a video detection range; and
correcting the initial range bin based on the video detection range to obtain the target range bin.

8. The measurement method according to claim 5, wherein said performing the signal value prediction based on the individual feature representation data, the environmental feature representation data, and the vital sign spectrum data to obtain the vital sign signal value with the differential impact removed comprises:
obtaining continuous multi-frame image data;
obtaining second feature data based on the continuous multi-frame image data;
performing feature fusion on the vital sign spectrum data of the measured object and the second feature data to obtain a fused feature, the vital sign spectrum data of the measured object being obtained based on the digital mixing signal of the first measurement device; and
predicting a vital sign signal value based on the individual feature representation data, the environmental feature representation data, and the fusion feature to obtain the vital sign signal value with the differential impact removed.

9. The measurement method according to claim 8, wherein:
the multi-frame image data is RGB-encoded data; and
said obtaining the second feature data based on the multi-frame image data comprises:
determining a region of interest in the multi-frame image data, the region of interest comprising a facial region;
cropping the multi-frame image data based on the determined region of interest to obtain a corresponding plurality of pieces of region-of-interest image data;
converting the plurality of pieces of region-of-interest image data from the RGB-encoded data into YUV-encoded data to obtain a corresponding plurality of pieces of region-of-interest chromaticity data; and
obtaining the second feature data based on the plurality of pieces of region-of-interest chromaticity data.

10. The measurement method according to claim 9, wherein said obtaining the second feature data based on the plurality of pieces of region-of-interest chromaticity data comprises:
extracting a remote photoplethysmography signal from the plurality of pieces of region-of-interest chromaticity data; and
obtaining the second feature data based on the remote photoplethysmography signal.

11. The measurement method according to claim 1, wherein said performing the signal value prediction based on the individual feature representation data, the environmental feature representation data, and the vital sign spectrum data to obtain the vital sign signal value with the differential impact removed comprises:
performing feature combination on the individual feature representation data, the environmental feature representation data, and the vital sign spectrum data to obtain a feature combination result; and
performing the signal value prediction based on the feature combination result to obtain the vital sign signal value, the vital sign signal value comprising at least one of a heart rate or a respiratory rate.

12. The measurement method according to claim 11, wherein said performing the feature combination on the individual feature representation data, the environmental feature representation data, and the vital sign spectrum data to obtain the feature combination result comprises:
performing concatenation processing on the individual feature representation data, the environmental feature representation data, and the vital sign spectrum data to obtain the feature combination result.

13. The measurement method according to claim 1, wherein said obtaining the individual feature representation data of the measured object and the environmental feature representation data of the environment where the measured object is located comprises:
obtaining a video signal by capturing the environment where the measured object is located;
determining, based on the video signal, environmental data and an individual recognition result of the measured object;
performing mapping processing on the environmental data to obtain the environmental feature representation data of a predetermined dimension; and
performing the mapping processing on the individual recognition result to obtain the individual feature representation data of the predetermined dimension.

14. The measurement method according to claim 1, wherein the vital sign signal value is outputted by a target classification model, the target classification model being obtained by the following training process:
constructing a training sample set, wherein the training sample set comprises a plurality of training samples, each of the plurality of training samples comprising historical vital sign spectrum data, historical individual feature representation data, and historical environmental feature representation data, and wherein a label of each of the plurality of training samples adopts a historical vital sign signal truth value; and
training an initial classification model based on the plurality of training samples and the labels to obtain the target classification model.

15. The measurement method according to claim 14, wherein said constructing the training samples comprises:
determining the historical vital sign spectrum data based on a historical digital mixing signal collected by a first measurement device for the measured object or an object other than the measured object at a historical moment;
obtaining a historical video signal collected at the historical moment;
determining the historical individual feature representation data and the historical environmental feature representation data based on the historical video signal; and
taking the historical vital sign signal true value collected by a second measuring device for the measured object or the object other than the measured object at the historical moment as the label.

16. The measurement method according to claim 15, wherein:
the second measurement device is a measurement device different from the first measurement device; and
the second measurement device is any one of a mechanical measurement device or a biological signal measurement device.

17. A computer device, comprising:
a memory having a computer program stored thereon; and
a processor, wherein the processor is configured to implement, when executing the computer program, a human factor intelligence-based vital sign signal measurement method,
wherein the human factor intelligence-based vital sign signal measurement method comprises:
obtaining individual feature representation data of a measured object, environmental feature representation data of an environment where the measured object is located, and vital sign spectrum data of the measured object, wherein the individual feature representation data and the environmental feature representation data have a differential impact on the vital sign spectrum data; and
performing signal value prediction based on the individual feature representation data, the environmental feature representation data, and the vital sign spectrum data to obtain a vital sign signal value with the differential impact removed.

18. The computer device according to claim 17, wherein said obtaining the individual feature representation data of the measured object comprises:
obtaining biometric feature data of the measured object based on a video signal, the video signal being obtained by capturing the environment where the measured object is located; and
performing individual recognition on the measured object based on the biometric feature data in the video signal to obtain the individual feature representation data.

19. An edge computing device, comprising
a memory having a computer program stored thereon;
a processor; and
a communication interface,
wherein the processor is configured to implement, when executing the computer program, a human factor intelligence-based vital sign signal measurement method,
wherein the human factor intelligence-based vital sign signal measurement method comprises:
obtaining individual feature representation data of a measured object, environmental feature representation data of an environment where the measured object is located, and vital sign spectrum data of the measured object, wherein the individual feature representation data and the environmental feature representation data have a differential impact on the vital sign spectrum data; and
performing signal value prediction based on the individual feature representation data, the environmental feature representation data, and the vital sign spectrum data to obtain a vital sign signal value with the differential impact removed.

20. A computer-readable storage medium, having a computer program stored thereon, wherein the computer program is configured to implement, when executed by a processor, the vital sign signal measurement method according to claim 1.
